(19) United States
(12) Patent Application Publication
(10) Pub. No.: US 2025/0281224 A1
(43) Pub. Date: Sep. 11, 2025

(54) INTEGRATED HANDPIECE PLUG

(71) Applicant: TENTECH Inc., Seoul (KR)

(72) Inventors: Ki Hang KIM, Seoul (KR); Min Ji CHOI, Seoul (KR)

(73) Assignee: TENTECH Inc., Seoul (KR)

(21) Appl. No.: 18/289,756

(22) PCT Filed: Jan. 4, 2022
(86) PCT No.: PCT/KR2022/000105

§ 371 Date: Apr. 29, 2024

(30) Foreign Application Priority Data
May 10, 2021 (KR) 10-2021-0059956

(51) Int. Cl. A61B 18/12; A61B 18/00; A61B 90/00

(52) U.S. Cl. CPC A61B 18/12; A61B 90/08; A61B 2018/00005; A61B 2018/00083; A61B 2018/00178; A61B 2090/0808

(57) ABSTRACT

A handpiece plug includes a refrigerant supply part for supplying a gaseous refrigerant to the handpiece, a power connection part that transmits power to the handpiece, a control signal connection part that transmits a control signal for driving the handpiece, and a corresponding coupling part mechanically correspondingly coupled to a coupling part formed in a handpiece connector so as to be fixed on a main machine, wherein the refrigerant supply part, the power connection part, the control signal connection part, and the corresponding coupling part are integrally formed.

### TECHNICAL FIELD

The present disclosure relates to a handpiece plug to connect a main machine and a handpiece of radio frequency medical equipment.

### BACKGROUND ART

Human skin consists of layers of the stratum corneum, epidermis, dermis, and hypodermis, and the functions of the skin deteriorate due to aging and the action of ultraviolet rays.

As interest in skin increases, a variety of skin care devices related to skin treatments are being developed, and at the same time, many cosmetic dermatology hospitals and skin treatment clinics are emerging.

The most widely known skin treatment method recently is radio frequency (RF) therapy, in which radio frequency energy is applied to skin to induce local thermal damage within the skin to create microscopic wounds in order to promote skin regeneration from the epithelium to the dermis, and the microscopic wounds stimulate the release of cell growth factors to maximize natural healing and regeneration of the skin.

In general, a handpiece is used to apply radio frequency waves to a desired treatment area, and the handpiece is connected to a main machine with individual cables to receive control signals to control the operation of the handpiece, power for operation, and refrigerant to cool the handpiece and the recipient's skin.

In this way, as individual cables are provided to receive control signals, power, refrigerant, etc., it is cumbersome to connect a handpiece to a main machine and detach the handpiece after use.

Moreover, such cable is provided in a fairly generous length so that an operator can freely position a handpiece, and is wrapped in a thick sheath for safety, which makes the cable quite heavy. Accordingly, the operator may feel considerable fatigue from operating the handpiece for a long time, and it is difficult to precisely position the handpiece on a treatment area and move the handpiece on the recipient's skin.

### DISCLOSURE

### Technical Problem

The present disclosure has been made to solve the above problems, and an objective of the present disclosure is to provide a handpiece plug that allows easy attachment and removal of a handpiece.

### Technical Solution

In order to achieve the above mentioned objective, there is provided an integrated handpiece plug. The handpiece plug includes: a refrigerant supply part for supplying a gaseous refrigerant to the handpiece; a power connection part that transmits power to the handpiece; a control signal connection part that transmits a control signal for driving the handpiece; and a corresponding coupling part mechanically correspondingly coupled to a coupling part formed in a handpiece connector so as to be fixed on a main machine, wherein the refrigerant supply part, the power connection part, the control signal connection part, and the corresponding coupling part may be integrally formed.

According to an embodiment of the present disclosure, the power connection part and the control signal connection part may be arranged to be spaced apart from each other in left and right directions.

According to an embodiment of the present disclosure, an insulating member may be positioned between the power connection part and the control signal connection part.

According to an embodiment of the present disclosure, the corresponding coupling part may be provided on an outside of the power connection part and on an outside of the control signal connection part, and the corresponding coupling part may be provided in different numbers on a first side and a second side of a front of a body of the handpiece plug, so that the handpiece plug may be asymmetrical in the left and right directions.

According to an embodiment of the present disclosure, the corresponding coupling part may be provided in an even number on the first side and in an odd number on the second side.

According to an embodiment of the present disclosure, the refrigerant supply part may be provided at a position spaced apart from the power connection part or the control signal connection part in a vertical direction, so that the handpiece plug may have an asymmetrical shape with respect to the vertical direction.

According to an embodiment of the present disclosure, the coupling part and the corresponding coupling part may each have either a shape with a protruding guide protrusion or a shape with a guide groove.

### Advantageous Effects

According to the present disclosure, by using an integrated handpiece plug, a handpiece and a main machine can be connected with a unified cable.

Furthermore, when an operator performs a procedure using a handpiece, usability can be improved by reducing the weight of the handpiece.

### DESCRIPTION OF DRAWINGS

FIG. 1 is a plan view showing a handpiece plug according to an embodiment of the present disclosure;

FIG. 2 is a perspective view showing a handpiece plug according to an embodiment of the present disclosure;

FIG. 3 is a plan view showing a main machine according to an embodiment of the present disclosure;

FIG. 4 is a plan view showing a handpiece connector according to an embodiment of the present disclosure; and

FIG. 5 is a perspective view showing a handpiece connector according to an embodiment of the present disclosure.

### BEST MODE

Similar r reference numbers in the drawings refer to identical or similar functions across various aspects.

Hereinafter, specific details for carrying out the present disclosure will be described based on embodiments reference to the drawings, and these embodiments are described in sufficient detail to enable those skilled in the art to practice the present disclosure.

It should be understood that the various embodiments of the present disclosure are different from one another but are not necessarily mutually exclusive. For example, specific shapes, structures, and characteristics described herein with respect to one embodiment may be implemented in other embodiments without departing from the spirit and scope of the present disclosure.

Therefore, the detailed description described below is not intended to be taken in a limited sense, and the scope of the present disclosure is limited solely by the appended claims, together with all equivalents to what those claims assert if properly described.

As an example, radio frequency medical equipment according to an embodiment of the present disclosure is a skin care device for procedures such as lifting or tightening by emitting radio frequency waves to the skin of a recipient. In addition, the radio frequency medical equipment may be one of a variety of known medical devices including handpieces, such as a skin treatment device using ultrasound, a skin treatment device using a laser, etc. A main machine of such radio frequency medical equipment may be provided with: a moving means installed at the lower part thereof and having a plurality of wheels for movement; and a display means on the upper part thereof for displaying operation and procedure status information for treatment and beauty performed by an operator by driving a handpiece. In addition, the main machine may accommodate electrical and electronic components, pumps, etc. for driving the handpiece.

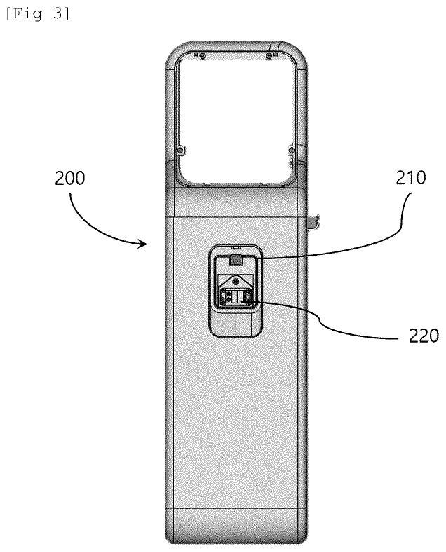

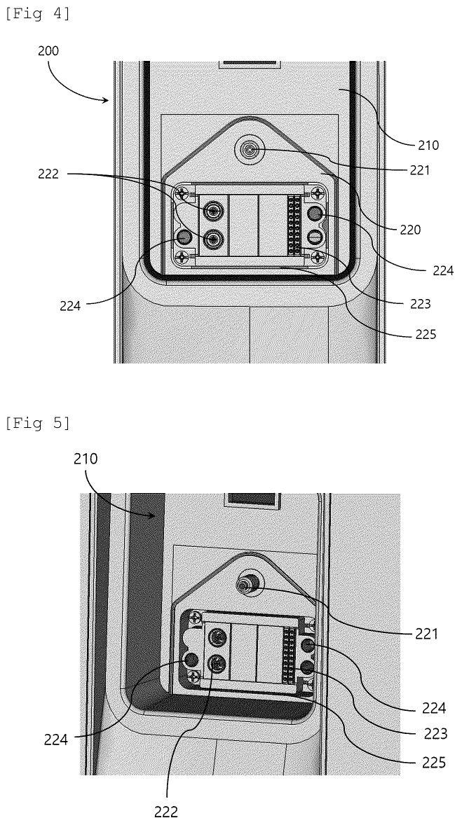

Referring to FIGS. 3 to 5, a main machine 200 of radio frequency medical equipment may have a depression part 210 having a predetermined depth on the front surface thereof. A handpiece connector 220 is provided on the bottom surface of the depression part 210.

The handpiece and the main machine 200 are mechanically and electrically connected through a cable. Mechanical and electrical connection is made by connecting an integrated handpiece plug 100 provided on the handpiece side to the handpiece connector 220 provided on the main machine 200 side. Since interconnection is made, it is preferable that connection terminals (a refrigerant supply part 110, a power connection part 120, a control signal connection part 130, and a corresponding coupling part 140) provided on the integrated handpiece plug 100 and connection terminals (a refrigerant supply connector part 221, a power connection connector part 222, a control signal connection connector part 223, and a coupling part 224) provided on the handpiece connector 220 have complementary shapes. In this case, having complementary shapes may mean that, for example, when the coupling part 224 is provided in a male shape with a protruding guide protrusion, the corresponding coupling part 140 is provided in a female shape with a guide groove into which the guide protrusion may be inserted.

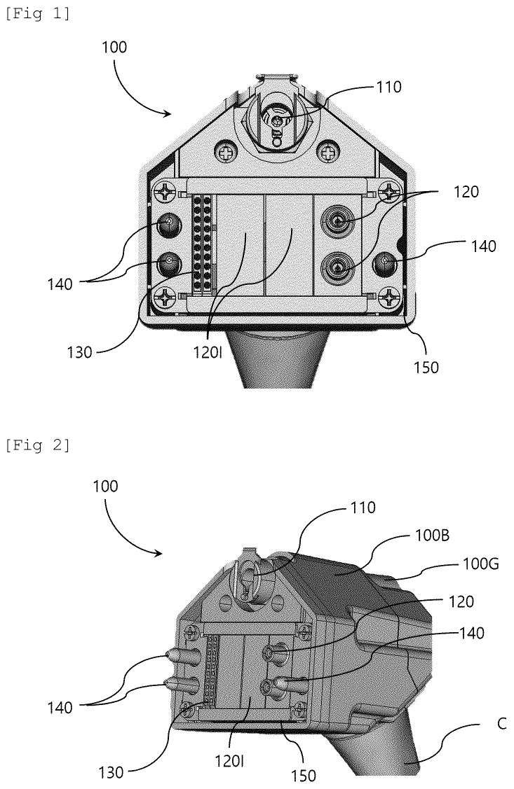

Hereinafter, an integrated handpiece plug according to an embodiment of the present disclosure will be described with reference to FIGS. 1 and 2.

The integrated handpiece plug according to the present disclosure has a body 100B on which a gripping body 100G is provided, and is coupled with a cable C connected to the end of the body 100B. A plurality of connection terminals connected to the handpiece connector are provided on the front of the body 100B.

To be specific, the integrated handpiece plug 100 includes: the refrigerant supply part 110 connected to the refrigerant supply connector part 221 to supply a gaseous refrigerant to the handpiece; the power connection part 120 connected to the power connection connector part 222 to transmit power to the handpiece; the control signal connection part 130 connected to the control signal connection connector part 223 to transmit a control signal for driving the handpiece; and the corresponding coupling part 140 mechanically correspondingly coupled to the coupling part 224 formed in the handpiece connector 220 so as to be fixed on the main machine 200. The refrigerant supply part 110, the power connection part 120, the control signal connection part 130, and the corresponding coupling part 140 are all integrally formed on the front of the body 100B, and a fitting groove 150 is formed along the outer periphery of the front of the body 100B.

The power connection part 120 and the control signal connection part 130 are arranged to be spaced apart from each other in the left and right directions, and an insulating member 120I is positioned therebetween.

The corresponding coupling part 140 is provided on the outside of the power connection part 120 and on the outside of the control signal connection part 130. Preferably, the number of the corresponding coupling parts 140 provided on one side and the number of the corresponding coupling parts 140 provided on the other side are different, so that the shape is asymmetrical in the left and right directions. In the embodiment of the present disclosure, the number of the corresponding coupling parts 140 provided on one side is even, and the number of the corresponding coupling parts 140 provided on the other side is odd.

Meanwhile, the refrigerant supply part 110 is provided at a position spaced apart from the power connection part 120 or the control signal connection part 130 in the vertical direction. Accordingly, the integrated handpiece plug 100 has an asymmetrical shape with respect to the vertical direction.

As described above, the present disclosure has been described with specific details such as specific components, limited embodiments, and drawings, but these are provided only to facilitate a more general understanding of the present disclosure, and the present disclosure is not limited to the above embodiments. Various modifications and variations may be made from the above description by those skilled in the art to which the present disclosure pertains.

Therefore, the spirit of the present disclosure should not be limited to the described embodiments. Not only the scope of the patent claims described below, but also all things that are equivalent or have equivalent modifications to the scope of these claims fall within the scope of the present disclosure.

1. In radio frequency medical equipment in which a handpiece connector is installed in a depression part provided on a surface of a main machine, and in which the handpiece connector and a handpiece are mechanically and electrically connected to each other through an integrated handpiece plug, the handpiece plug comprising:
a refrigerant supply part for supplying a gaseous refrigerant to the handpiece;
a power connection part that transmits power to the handpiece;
a control signal connection part that transmits a control signal for driving the handpiece; and
a corresponding coupling part mechanically correspondingly coupled to a coupling part formed in the handpiece connector so as to be fixed on the main machine,
wherein the refrigerant supply part, the power connection part, the control signal connection part, and the corresponding coupling part are integrally formed.

2. The integrated handpiece plug of claim 1, wherein the power connection part and the control signal connection part are arranged to be spaced apart from each other in left and right directions.

3. The integrated handpiece plug of claim 2, wherein an insulating member is positioned between the power connection part and the control signal connection part.

4. The integrated handpiece plug of claim 2, wherein the corresponding coupling part is provided on an outside of the power connection part and on an outside of the control signal connection part, and the corresponding coupling part is provided in different numbers on a first side and a second side of a front of a body of the handpiece plug, so that the handpiece plug is asymmetrical in the left and right directions.

5. The integrated handpiece plug of claim 4, wherein the corresponding coupling part is provided in an even number on the first side and in an odd number on the second side.

6. The integrated handpiece plug of claim 1, wherein the refrigerant supply part is provided at a position spaced apart from the power connection part or the control signal connection part in a vertical direction, so that the handpiece plug has an asymmetrical shape with respect to the vertical direction.

7. The integrated handpiece plug of claim 1, wherein the coupling part and the corresponding coupling part each have either a shape with a protruding guide protrusion or a shape with a guide groove.
